(19) United States
(12) Patent Application Publication
(10) Pub. No.: US 2005/0137808 A1
(43) Pub. Date: Jun. 23, 2005

(54) Method for conceptualizing protein interaction networks using gene ontology

(76) Inventors: Jae Hun Choi, Daejeon-Shi (KR); Seon Hee Park, Daejeon-Shi (KR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/971,872

(22) Filed: Oct. 22, 2004

(30) Foreign Application Priority Data
Dec. 18, 2003 (KR) 2003-92794

(51) Int. Cl. G06F 19/00; G01N 33/48; G01N 33/50

(52) U.S. Cl. 702/19

(57) ABSTRACT

Provided is a method for conceptualizing protein interaction networks. The method conceptualizes and simplifies complicated and enormous protein interaction networks wherein the method comprises the steps of (a) conceptualizing protein nodes that form the protein interaction network as gene ontology concepts to reconfigure the network; (b) integrating nodes including the same concepts in the reconfigured network into one node to generate the network by means of exact match; and (c) integrating several nodes having similar concepts in the generated network into one node to reconfigure the generated network by means of approximate match.

### BACKGROUND

1. Field of the Invention

The present invention relates to a method for conceptualizing protein interaction networks, and more particularly, to a method for simply conceptualizing complicated and enormous protein interaction networks which have visualized a protein interaction relation present within a living body using gene ontology to allow it to be effectively visualized in various viewpoints while allowing biologists to better understand it.

2. Discussion of Related Art

In general, protein interaction networks are used as important information for identifying a biological function that the protein has in a whole viewpoint, because an unknown function of a specific protein may be inferred from other protein interacting with the specific protein in the protein interaction networks.

In other words, the protein capable of suppressing or activating the specific function may be predicted. The protein interaction networks using such properties are used as significantly important information in determining target protein development ranged from new drug to high value added. To that end, a system must visualize views in various viewpoints so as to allow a user to analyze interaction networks having enormous proteins from various angles.

In the related art, the interaction networks with respect to specific proteins are visualized with views as follows. To detail this, the interaction networks are represented with binary relations between proteins, and these binary relations are visualized in a network form by means of a conventional graph visualization algorithm.

In this case, nodes of the visualized networks indicate a protein name or a gene name, and links for lining these nodes indicate an interaction relation between two proteins. In addition, Force-Directed Placement (FDP) is widely used as the network visualization algorithm.

The amount of relations between proteins are so large in a living body, which causes the user to have a difficulty in understanding the network and also causes the network not to be analyzed in various viewpoints when the network is visualized with such conventional views.

### SUMMARY OF THE INVENTION

The present invention is directed to a method for simply conceptualizing complicated and enormous protein interaction networks which have visualized a protein interaction relation in bioinformatics using three properties (CC, BP, MF) of gene ontology to allow it to be effectively visualized in various viewpoints while allowing biologists to better understand it.

One aspect of the present invention is to provide a method for simply conceptualizing a complicated protein interaction network using gene ontology, which comprises the steps of: (a) conceptualizing protein nodes that form the protein interaction network as gene ontology concepts to reconfigure the network; (b) integrating nodes including the same concepts in the reconfigured network into one node to generate the network by means of exact match; and (c) integrating several nodes having similar concepts in the generated network into one node to reconfigure the generated network by means of approximate match.

### BRIEF DESCRIPTION OF THE DRAWINGS

FIG. 1 views a schematic block configuration of a hardware system for implementing a method for conceptualizing protein interaction networks using gene ontology in accordance with one embodiment of the present invention.

FIG. 2 is a flow chart for explaining a method for conceptualizing protein interaction networks using gene ontology in accordance with one embodiment of the present invention.

FIG. 3 is a detailed flow chart for explaining the protein conceptualization procedure of FIG. 2.

FIG. 4 is a detailed flow chart for explaining the network conceptualization procedure by means of exact match of FIG. 2.

FIG. 5 is a detailed flow chart for explaining the network conceptualization procedure by means of approximate match of FIG. 2.

FIG. 6 is a partial view of a gene ontology database (DB) applied to one embodiment of the present invention.

FIG. 7 is a view for explaining a method for conceptualizing protein interaction networks using gene ontology in accordance with one embodiment of the present invention.

### DETAILED DESCRIPTION OF PREFERRED EMBODIMENTS

The present invention will now be described more fully hereinafter with reference to the accompanying drawings, in which preferred embodiments of the invention are shown. This invention may, however, be embodied in different forms and should not be construed as limited to the embodiments set forth herein.

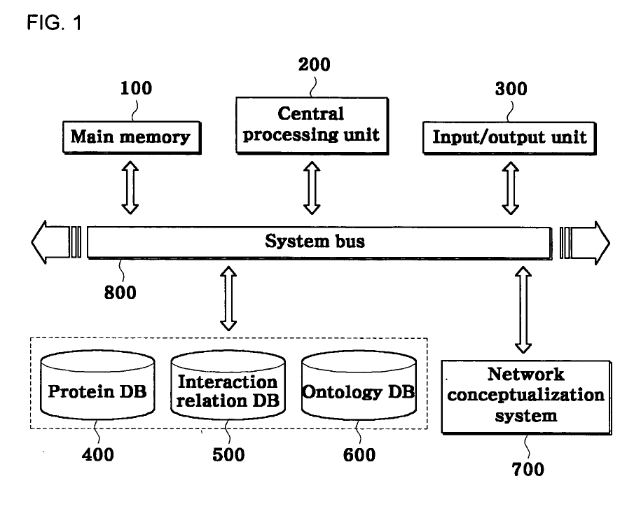

FIG. 1 views a schematic block configuration of hardware system for implementing the method for conceptualizing protein interaction networks using gene ontology in accordance with one embodiment of the present invention.

As shown in FIG. 1, a hardware system for implementing a method for conceptualizing protein interaction networks using gene ontology in accordance with one embodiment of the present invention is comprised of a main memory 100, a central processing unit 200, an input/output unit 300, a protein DB 400, an interaction network DB 500, an ontology DB 600, a network conceptualization system 700, and a system bus 800.

In the above-mentioned configuration, information with respect to the protein DB 400, the interaction network DB 500, and the ontology DB 600, which are required for each step and the network conceptualization system 700, are loaded in the main memory 100.

In this case, information of the protein DB 400 may use SWISS-PROT, information of the interaction network DB 500 may use DIP or BIND, and information of the ontology DB 600 may use Gene Ontology.

The central processing unit 200 acts to perform information of the network conceptualization system 700 loaded in the main memory 100 on a step basis.

The input/output unit 300 receives information necessary for the system from a user and outputs, on a screen, contents related with the network automatically conceptualized by the system. In this case, messages or information among components shown in FIG. 1 are transceived through the system bus 800.

Hereinafter, a method for conceptualizing the protein interaction networks using the gene ontology having the above-mentioned configuration of the present invention will be described in detail.

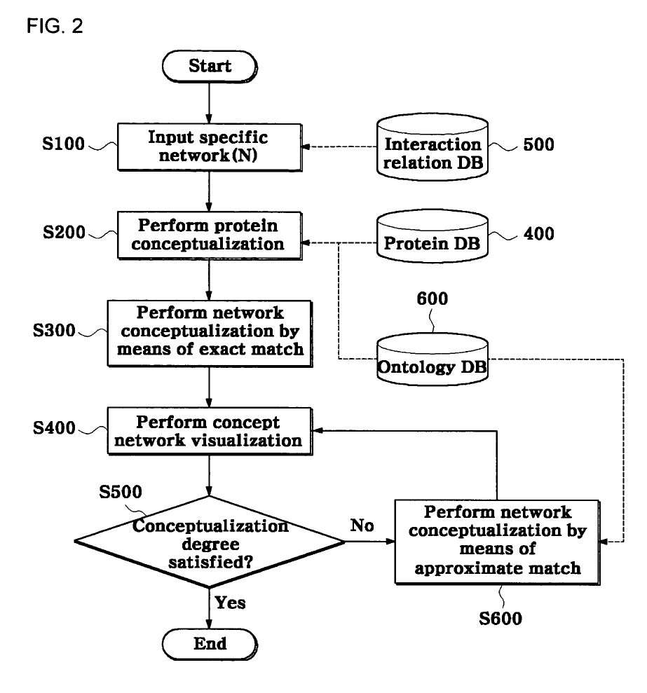

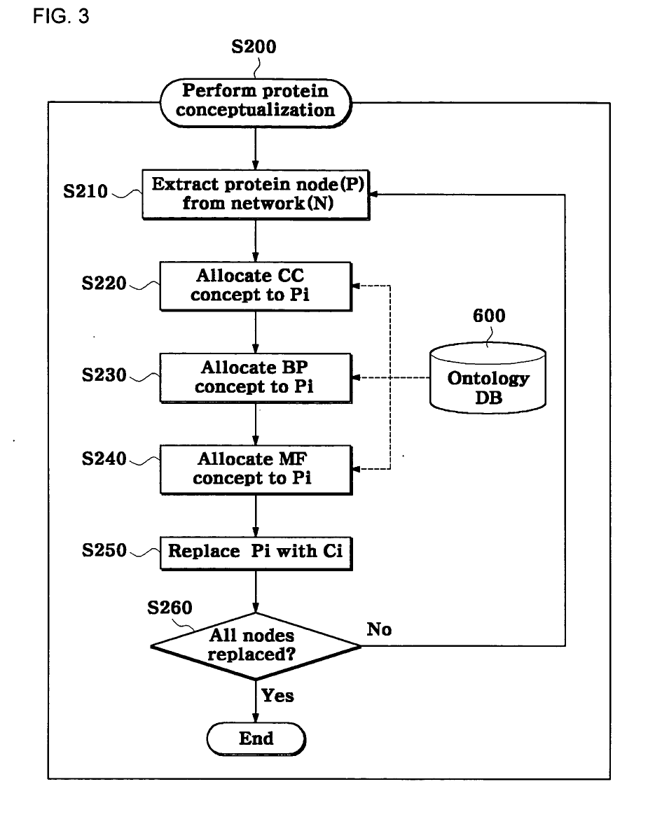

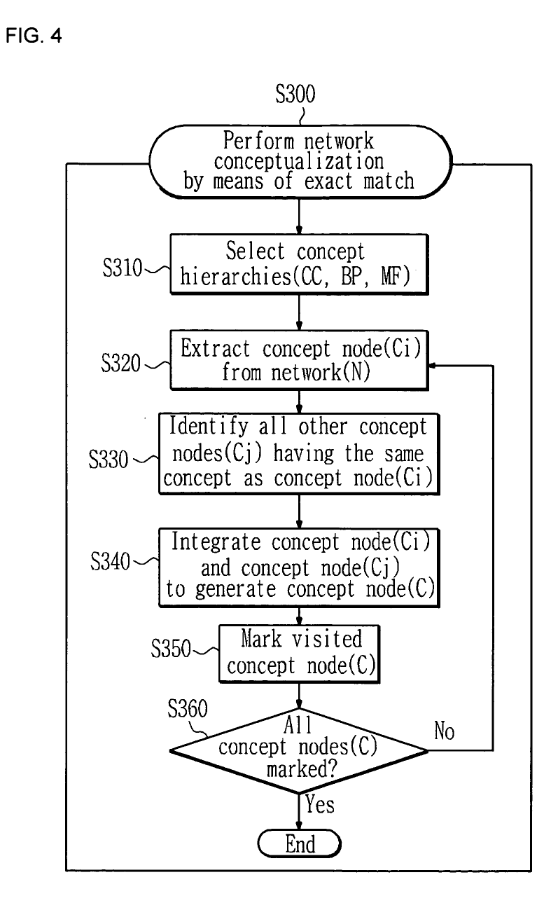

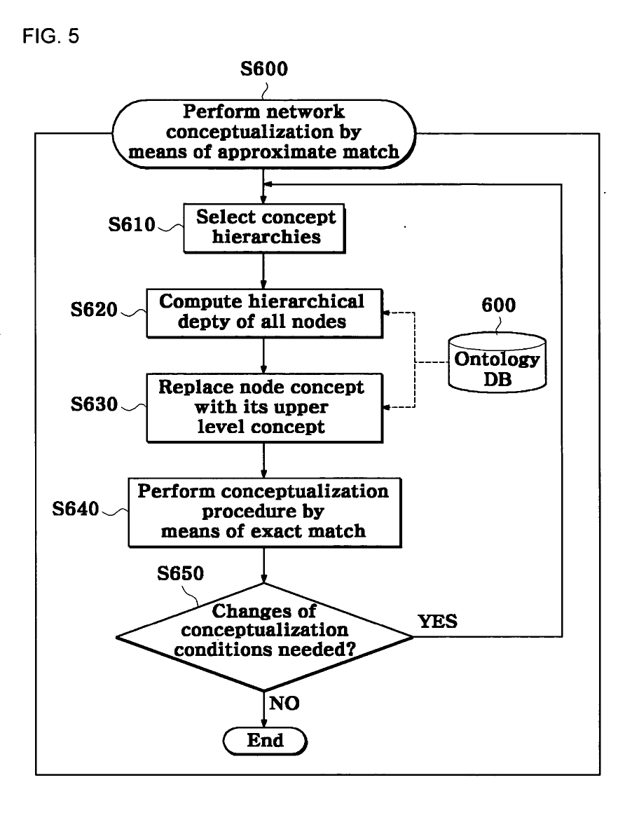

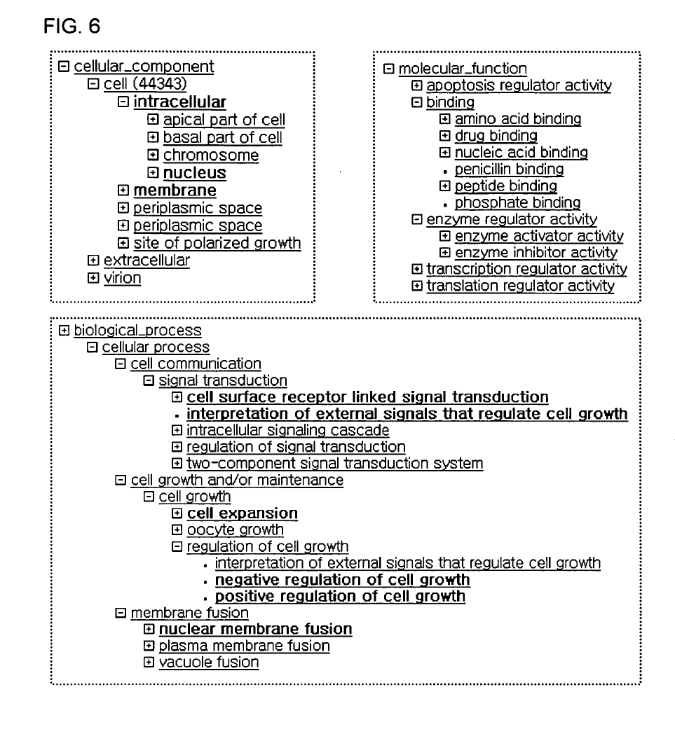

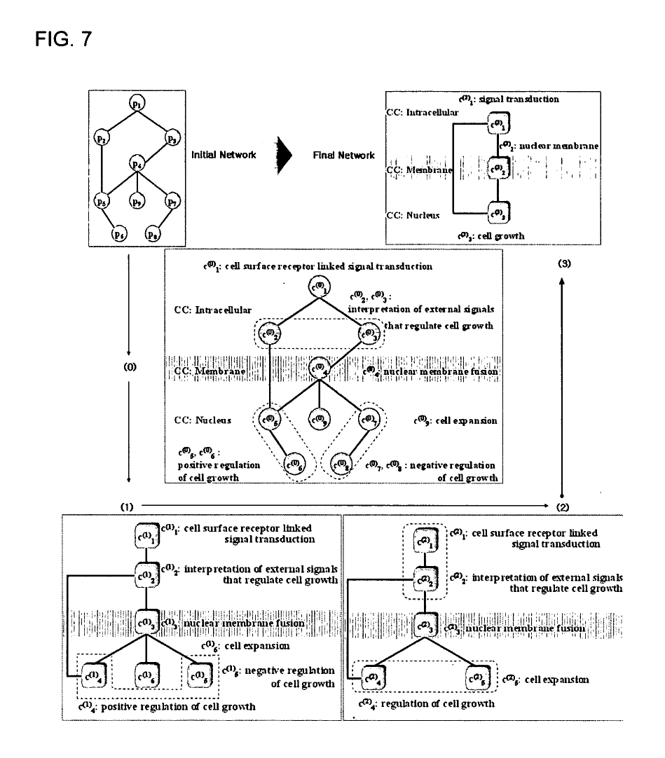

FIG. 2 is a flow chart for explaining a method for conceptualizing protein interaction networks using gene ontology in accordance with one embodiment of the present invention, FIG. 3 is a detailed flow chart for explaining the protein conceptualization procedure of FIG. 2, FIG. 4 is a detailed flow chart for explaining the network conceptualization procedure by means of exact match of FIG. 2, FIG. 5 is a detailed flow chart for explaining the network conceptualization procedure by means of approximate match of FIG. 2, FIG. 6 is a partial view of a gene ontology database (DB) applied to one embodiment of the present invention, and FIG. 7 is a view for explaining a method for conceptualizing protein interaction networks using gene ontology in accordance with one embodiment of the present invention.

As shown in FIG. 2 to FIG. 7, a specific network (N) is input from the interaction network DB 500 in the step S100, and nodes of proteins pertained in the specific network (N) are identified from the protein DB 400 in the step S200, and these proteins are replaced with concepts of the ontology DB 600 consisting of three hierarchies, namely, Cellular Component (hereinafter referred to as “CC”), Biological Process (hereinafter referred to as “BP”), and Molecular Function (hereinafter referred to as “MF”), so that the network is reconfigured.

Next, nodes having the same concepts among the nodes included in the reconfigured network are integrated as one node in the step S300. In this case, relation information is also integrated with the one node to conceptualize the network. In other words, the network conceptualization is performed by means of exact match.

Next, the reconfigured network is automatically visualized by applying a Force-Directed Placement (FDP) algorithm in the step S400, and a conceptualization degree of the visualized network is compared to a preset reference degree in the step S500, and terminates when it is satisfied, or proceeds to the step S600 when it is not satisfied to thereby integrate nodes having similar concepts among the nodes included in the reconfigured network into one node. In other words, the network conceptualization is performed by means of approximate match and the process returns to the step S400.

In this case, relation information is also integrated into the one node to conceptualize the network. Similarity between these concepts is identified using concept hierarchies of the ontology DB 600. Since these similar nodes are integrated into one node, the conceptualized network may be visualized by means of the step S400.

In the meantime, to detail the protein conceptualization procedure in the step S200 with reference to FIG. 3, one protein is responsible for some functions of specific biological processing in a specific portion of a cell in the interaction network. These protein properties may be exhibited as concepts present in the CC, BP, and MF hierarchies of the gene ontology.

As shown in FIG. 3, one protein node (e.g., Pᵢ) is extracted from the network (N) in the step S210, and CC, BP, MF concepts corresponding to the protein node (Pᵢ) are allocated from the ontology DB 600 in the steps S220, S230, and S240, respectively. In this case, an “unknown” value is allocated to protein of which each concept is not known.

The protein node (Pᵢ) is replaced with a concept node (Cᵢ) in the step S250.

To detail this with reference to FIG. 7, Pᵢ of the first network is replaced with C₁⁽⁰⁾ by allocating CC concept “intracellular”, BP concept “cell surface receptor linked signal transduction”, and MF concept “Unknown”. P₂ and P₃ are replaced with C₂⁽⁰⁾ by allocating CC concept “intracellular”, BP concept “interpretation of external signals that regulate cell growth”, and MF concept “Unknown”. Proteins (P₃ . . . ₄) are also conceptualized by means of such method to thereby generate a protein conceptualization network. Thus, for simplicity of description, CC and BP hierarchies are employed to describe the network conceptualization procedure in the present embodiment.

To detail the network conceptualization procedure by means of exact match in the step S300 with reference to FIG. 4, respective nodes in the network where proteins are conceptualized are exhibited as CC, BP, and MF concepts. As a result, nodes exhibited with the same concepts may be present in the network.

As shown in FIG. 4, some concept hierarchies (CC, BP, and MF) are selected in the gene ontology to proceed the conceptualization by means of exact match in the step S310, and one concept node (Cᵢ) is extracted from the network (N) in the step S320.

Next, all other concept nodes (C_{j,j=1, . . . ,n}) having the same concept as the concept node (Cᵢ) are identified in the step S330. In this case, only the gene ontology concepts corresponding to the hierarchies selected in the step S310 are subject to comparison.

Subsequently, the identified concept nodes (C_{j,j=1, . . . ,n}) and the extracted concept node (Cᵢ) are integrated and replaced with one concept node (C) in the step S340. In this case, all relations that the concept node (Cᵢ) and the concept node (Cⱼ) have are also integrated with the concept node (C), so that the meaning of the network (N) still remains the same.

Next, after the concept node (C) is marked so as not to visit the concept node (C) again in the step S350, it is determined whether all concept nodes (C) are visited in the step S360, and the procedure returns to the step S320 when there exists node(s) to be visited.

To detail this with reference to FIG. 7, the network (0) represents the conceptualization procedure by means of exact match of the network (1), and there are no other nodes having the concept such as C₁⁽⁰⁾, so that the node (Cᵢ⁽¹⁾) of the network (1) is mapped as it is. Nodes (C₂⁽⁰⁾, C₃⁽⁰⁾) have CC and BP concepts corresponding to “intracellular” and “interpretation of external signals that regulate cell growth”, respectively, so that they are integrated to the node (C₂⁽¹⁾) of the network (1). Nodes (C₅⁽⁰⁾, C₆⁽⁰⁾) have “nucleus” and “positive regulation of cell growth”, respectively, by means of such method, so that they are integrated to C₄⁽¹⁾ of the network (1). In this case, since the node (C₂⁽¹⁾) has a relation with C₁⁽¹⁾ and C₄⁽¹⁾, which means that this node also has the relation integrated with those of the two nodes (C₂⁽⁰⁾, C₃⁽⁰⁾).

To detail the network conceptualization procedure by means of approximate match with reference to FIG. 5 in the above-mentioned step S600, gene ontology concepts included in the network nodes may have similar meaning from one another. Thus, nodes including closely related concepts from one another are also integrated into one node, which leads to better conceptualize the networks.

As shown in FIG. 5, one gene ontology hierarchy for performing conceptualization by means of approximate match is selected in the step S610, and depths of concepts that each of all nodes has are computed in the step S620. In this case, the hierarchical depth of the concept is evaluated in the gene ontology hierarchy selected in the step S610.

Next, concepts of the node having the deepest depth among the computed nodes are replaced with their one level higher concept in the step S630, and the procedure returns to the step S300 to perform network conceptualization with respect to nodes including the replaced concepts by means of exact match in the step S640.

Next, it is determined whether the conceptualization condition should be changed by a user in the step S650, and the procedure terminates when the user does not want to continue performing the conceptualization, or returns to the step S610 when the user want to.

Referring to FIG. 7, as conceptualization steps by means of approximate match from a network (1) to a network (2) and from the network (2) to a network (3), system receives information that the conceptualization hierarchy is BP from a user as in the step S610. Hierarchical depths of all nodes present in the network (1) are computed as in the step S620.

All BP concepts allocated to the C₁⁽¹⁾, C₂⁽¹⁾, and C₆⁽¹⁾ in the gene ontology BP hierarchy (See FIG. 6) have depths of 5 and C₃⁽¹⁾ has a depth of 4. In addition, depths of C₄⁽¹⁾ and C₅⁽¹⁾ are 6. Thus, concepts present in the C₄⁽¹⁾ and C₅⁽¹⁾ corresponding to “positive regulation of cell growth” and “negative regulation of cell growth”, respectively, are replaced with its upper concept “regulation of cell growth” with reference to the BP gene ontology as in the step S630.

These replaced C₄⁽¹⁾ and C₅⁽¹⁾ are replaced with C₄⁽²⁾ of the network (2) by means of a conceptualization procedure using exact match as in the step S630.

The network (2) may be conceptualized to be network (3) using such method. In other words, each hierarchical depth of C₁⁽²⁾, C₂⁽²⁾, C₄², and C₅⁽²⁾ is evaluated to be 5. Thus, concepts that these nodes have are replaced with their upper concepts. In other words, both of “cell surface receptor linked signal transduction” of C₁⁽²⁾ and “interpretation of external signals that regulate cell growth” of C₂⁽²⁾ are replaced with “signal transduction”, and both of “regulation of cell growth” of C₄⁽²⁾ and “cell expansion” of C₅⁽²⁾ are replaced with “cell growth”. By means of these replaced concepts employing exact match in the conceptualization procedure, the network (3) may be generated. Such procedure may be repeated to thereby generate a more simplified network resulted from enormous networks.

While the present invention has been described for the method for conceptualizing protein interaction networks using gene ontology with reference to a preferred embodiment, it is understood that the disclosure has been made for purpose of illustrating the invention by way of examples and is not limited to the scope of the invention. And one skilled in the art can make amend and change the present invention without departing from the scope and spirit of the invention.

In accordance with the method for conceptualizing the protein interaction networks using gene ontology of the present invention as mentioned above, enormous and complicated protein interaction networks which are visualized with respect to an interaction relation of proteins present in a living body by means of three properties (CC, BP, MF) that the gene ontology has, are simply conceptualized while their meanings remain the same, which allows biologists to better understand it and to effectively visualize it in various viewpoints, and allows users to conceptually understand the interaction networks, and not only provides collective environment of interest that the users want to analyze but also remarkably reduces cost for network analysis.

1. A method for conceptualizing a protein interaction network using gene ontology, the method comprising the steps of:
(a) conceptualizing protein nodes that form the protein interaction network as gene ontology concepts to reconfigure the network;
(b) integrating nodes including the same concepts in the reconfigured network into one node to generate the network by means of exact match; and
(c) integrating several nodes having similar concepts in the generated network into one node to reconfigure the generated network by means of approximate match,
whereby the protein interaction network is changed from a complicated form into a simplified form.

2. The method as claimed in claim 1, wherein the step (a) includes the sub-steps of:
(a1) extracting one protein node (Pᵢ) from the network (N);
(a2) allocating CC, BP, and MF concepts corresponding to the extracted protein node (Pᵢ), respectively; and
(a3) replacing all protein nodes (Pᵢ) with a concept node (Cᵢ).

3. The method as claimed in claim 1, wherein the step (b) includes the sub-steps of:
(b1) selecting a plurality of concept hierarchies (CC, BP, MF) from the gene ontology;
(b2) extracting one concept node (Cᵢ) from the network (N);
(b3) identifying all other concept nodes (C_{j,j=1, . . . ,n}) having the same concept as the extracted concept node (Cᵢ);
(b4) integrating the extracted concept node (Cᵢ) and the identified concept nodes (C_{j,j=1, . . . ,n}) to generate one concept node (C); and
(b5) marking the all generated concept nodes (C).

4. The method as claimed in claim 1, wherein the step (c) includes the sub-steps of:
(c1) selecting one ontology hierarchy;
(c2) computing concept depths of all nodes based on the selected ontology hierarchy;
(c3) replacing node concepts having the deepest depth among the computed nodes with their upper concepts;
(c4) returning to the step (b) to perform the network conceptualization by means of the exact match with respect to nodes including the replaced concepts; and
(c5) repeating the steps (c1) to (c4) when a user wants to continue performing the conceptualization.
